(19) United States
(12) Patent Application Publication
(10) Pub. No.: US 2017/0189558 A1
(43) Pub. Date: Jul. 6, 2017

(54) ULTRASMALL LUMINESCENT NANOSENSORS COMPOSITIONS AND APPLICATIONS

(71) Applicant: The Board of Regents of the University of Texas System, Austin, TX (US)

(72) Inventors: Jie ZHENG, Allen, TX (US); Shasha SUN, Plano, TX (US); Xuhui NING, Dallas, TX (US)

(73) Assignee: The Board of Regents of the University of Texas System, Austin, TX (US)

(21) Appl. No.: 15/395,196

(22) Filed: Dec. 30, 2016

Related U.S. Application Data
(60) Provisional application No. 62/273,147, filed on Dec. 30, 2015.

(51) Int. Cl. A61K 49/00

(52) U.S. Cl. CPC A61K 49/0093; A61K 49/0041; A61K 49/005

(57) ABSTRACT

The present disclosure relates to the production and use of ultrasmall luminescent nanoparticles coupled to dyes and the obtained nanoparticles exhibit functionalities that can be highly sensitive to the local chemical environment changes. These nanoparticles may act as ultrasmall nanosensors to ratiometrically report pH or glutathione in the local environment.

The present application claims the benefit of priority to U.S. Provisional Application Ser. No. 62/273,147, filed on Dec. 30, 2015, the entirety of which is incorporated herein by reference.

### STATEMENT REGARDING FEDERALLY-SPONSORED RESEARCH OR DEVELOPMENT

This invention was made with government support under Grant No. R01DK103363 awarded by the National Institutes of Health. The government has certain rights in the invention.

### BACKGROUND

A. Field

The disclosure relates generally to the fields of medicine, diagnostics, therapy, and imaging. More particularly, it relates to the synthesis and use of pH-sensitive nanoparticles.

B. Related Art

Metal nanoparticles (NPs) generally do not fluoresce because of their large density of states; as a result, conjugation of organic dyes to non-luminescent metal NPs is essential for visualizing them in fluorescence imaging systems (Xie et al., 2010; Palner et al., 2015; Hu and Gao, 2010). For instance, Chan et al. conjugated organic dyes such as Cyto633 to different sized non-luminescent gold NPs (AuNPs), so that these AuNPs can be readily monitored in real time in vivo (Chou and Chan, 2012). In addition, due to surface plasmon absorption of metal nanoparticles, Raman scattering of organic dyes was significantly enhanced and has also found broad applications in bioimaging and sensing.

Different from conventional non-luminescent metal NPs, a new class of metal NPs that can give intrinsic fluorescence without being labeled with organic dyes has also emerged in the past decade (Zheng et al., 2003; Zheng et al., 2004; Zheng et al., 2007). By tuning particle size, crystallinity, surface ligands and valance states, the inventors and others were able to create a large number of luminescent metal NPs with tunable emission ranging from UV to NIR (Zhou et al., 2012; Zhou et al., 2011; Zheng et al., 2012). Complementary to dye-labeled non-luminescent metal NPs, metal NPs with intrinsic emissions have also found many applications in imaging and sensing. For instance, red-emitting AuNPs have been used to detect small metal ions, toxin and reactive oxygen species (Wei et al., 2010; Chen et al., 2013). More recent studies show that NIR-emitting AuNPs can serve as a new class of renal clearable contrast agents for rapid tumor diagnosis and kidney functional imaging (Liu et al., 2013; Yu et al., 2015; Yu and Zheng, 2015). While the emergence of these luminescent metal NPs suggests that organic dyes might no longer be necessary for detecting metal NPs in the fluorescence imaging systems, rational integration of organic dyes with ultrasmall metal nanoparticles can lead to a new class of nanosensors that can exhibit strong responses to external chemical or physical changes such as pH, glutathione, cysteine and light irradiation.

### SUMMARY

In accordance with the present disclosure, there is provided a composition comprising a nanoparticle, wherein the surface of the nanoparticle is conjugated with (a) a charged ligand susceptible to protonation or deprotonation by pH change and (b) a dye that is pH-insensitive when not conjugated with the nanoparticle. The nanoparticle may be a gold nanoparticle (i.e., only gold, predominantly gold, or partially gold). The nanoparticle may be about 0.5 nm to 10 nm in diameter, or may be about 1 nm to 5 nm in diameter, may be below 5 nm in diameter, below or less than 3 nm in diameter, below 1 nm in diameter, and optionally with a lower limit diameter of 0.5 nm, 0.6 nm, 0.7 nm, 0.8 nm, 0.9 nm or 1.0 nm. The nanoparticle metal may also comprise, consist of or consist essentially of silver, copper, platinum, or carbon. The nanoparticle may be luminescent, and may emit at blue to infrared wavelengths.

The nanoparticle may provide a ratiometric comparison of emissions from said dye and the luminescence. The charged ligand may be glutathione, cysteine, cysteine-glycine or cysteine-glutamate, or any other ligand that is differentially protonated across various pHs. The charged ligand may be capable of binding to at least one cellular component. The gold nanoparticle may comprise a second ligand capable of binding to at least one cellular component. The cellular component may be a tumor marker. The blood residence half-time of the nanoparticle after administration of the gold nanoparticle to a subject may be from about 2 hours to about 25 hours. The pH insensitive dye may be tetramethyl rhodamine (TAMRA), an Alexa Fluor Dyes, boron-dipyrromethene, or Rhodamine 6G. The composition may comprise two or more pH insensitive dye molecules. The two or more pH insensitive dye molecules may be different or the two or more pH insensitive dye molecules may be the same. The nanoparticle may be sensitive to a pH from about 5 to about 11, from about 6 to about 10, from about 7 to about 9, or at about pH 5, 6, 7, 8, 9, 10 or 11.

In another embodiment, there is provided a method for detecting pH of an environment comprising the steps of (a) contacting the environment with a composition comprising a nanoparticle as set out above; and (b) monitoring the emission from the nanoparticle by optical imaging, microscopic imaging or combinations thereof. The optical imaging technique may be fluorescence imaging or near infrared imaging. The environment may be in a living subject. The composition may be administered to said living subject orally, intravenously, nasally, subcutaneously, intramuscularly or transdermally. The monitoring may occur over time and detects a change in pH of the environment.

In yet another embodiment, there is provided a composition comprising a noble metal nanoparticle, wherein the surface of the nanoparticle is conjugated with (a) a charged ligand susceptible to protonation or deprotonation by pH change and (b) a pH-sensitive dye, wherein said pH-sensitive dye is more pH sensitive when conjugated to said nanoparticle than when not conjugated with the nanoparticle. The may be nanoparticle is about 0.5 nm to 10 nm in diameter, or about 1 nm to 5 nm in diameter. The nanoparticle may be luminescent, may be gold, and/or may emit at blue to infrared wavelengths. The nanoparticle may provide a ratiometric comparison of emissions from said dye and the luminescence. The charged ligand may be glutathione, cysteine, cysteine-glycine, cysteine-glutamate or any other ligand that is differentially protonated across various pHs. The charged ligand may also be capable of binding to at least one cellular component, such as a tumor marker. The nanoparticle may also comprise a second ligand capable of binding to at least one cellular component. The cellular component may be a tumor marker. The nanoparticle metal may also comprise, consist of or consist essentially of silver, copper, platinum, or carbon.

The blood residence half-time of the nanoparticle after administration of the gold nanoparticle to a subject may be from about 2 hours to about 25 hours. The pH sensitive dye may be 2-fold, 3-fold, 4-fold, 5-fold or 10-fold more sensitive when conjugated to gold nanoparticle as compared to its unconjugated state. The pH sensitive dye may be (6)-carboxyfluorescein, 7-hydroxycourmarin-3-carboxylic acid, rhodamine B octadecyl ester perchlorate, 2′,7′-Bis(3-carboxypropyl)-5(6)-carboxyfluorescein (BCECF), 8-hydroxypyrene-1,3,6-trisulfonic acid trisodium salt (HPTS). The nanoparticle may be sensitive to a pH from about 5 to about 11, from about 6 to about 10, from about 7 to about 9, or at about pH 5, 6, 7, 8, 9, 10 or 11. The nanoparticle may be 0.5-10 nm in diameter, or 1 nm to 5 nm in diameter. The nanoparticle may contain 2 dye particles.

In still yet another embodiment, there is provided a method for detecting pH of an environment comprising the steps of (a) contacting the environment with a composition comprising a nanoparticle as set out above; and (b) monitoring the emission from the nanoparticle by optical imaging, microscopic imaging or combinations thereof. The optical imaging technique may be fluorescence imaging or near infrared imaging. The environment may be in a living subject. The composition may be administered to said living subject orally, intravenously, nasally, subcutaneously, intramuscularly or transdermally. The monitoring may occur over time and detects a change in pH of the environment.

In yet another aspect, the present disclosure provides methods for determining the presence of a thiolated compound in an environment comprising the steps of:

(a) contacting the environment with a composition comprising a nanoparticle described herein; and

(b) monitoring the emission from the nanoparticle by optical imaging, microscopic imaging, or combinations thereof, wherein a change in the emission is associated with the presence of a thiolated compound.

The optical imaging technique may be fluorescence imaging or near infrared imaging. In some embodiments, the environment is in a living subject. The composition may be administered to said living subject orally, intravenously, nasally, subcutaneously, intramuscularly or transdermally. In some embodiments, monitoring occurs over time and detects a change in the concentration of the thiolated compound in the environment. In some embodiments, the thiolated compound is glutathione or cysteine.

Also provided is a method for detecting pH in a tumor environment in a subject comprising administering to the subject cell an effective amount of a composition as described above.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description.

It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-E. (FIG. 1A) Schematic illustration of the conjugation of pH insensitive tetramethyl-rhodamine (TAMRA) onto pH insensitive GS-AuNPs. (FIG. 1B) Agarose gel electrophoresis of GS-AuNPs (1), TG-AuNPs (2) and TAMRA (3). (FIG. 1C) Hr-TEM image and size distribution of TG-AuNPs. The scale bar is 10 nm. (FIG. 1D) Emission spectra of TAMRA, GS-AuNPs and TG-AuNPs collected at 350 nm excitation. (FIG. 1E) Absorption spectra of TAMRA, GS-AuNPs and TG-AuNPs.

FIGS. 2A-D. (FIG. 2A) Fluorescence spectra of TAMRA and GS-AuNPs at different pH values. (FIG. 2B) Fluorescence spectra of TG-AuNPs at different pH values. (FIG. 2C) The relationship between pH and I₅₈₇ nm/I₈₁₅ nm ratio. (FIG. 2D) Reversibility of fluorescence intensity ratios (I₅₈₇ nm/I₈₁₅ nm) of TG-AuNPs with pH change between 6 and 10.

FIGS. 3A-C. (FIG. 3A) Absorption spectra of TAMRA on TG-AuNPs at pH 6 and pH 10, respectively. (FIG. 3B) Zeta potentials of GS-AuNPs and TG-AuNPs at pH 6 and pH 10, respectively. (FIG. 3C) Schematic illumination of the pH responsive mechanism of TG-AuNPs.

FIGS. 4A-D. (FIG. 4A) Hr-TEM image and size distribution of FG-AuNPs. The scale bar is 10 nm. (FIG. 4B) Absorption spectra of 5(6)-carboxylfluorescein and 5(6)-carboxylfluorescein on FG-AuNPs. (FIG. 4C) Fluorescence spectra of FG-AuNPs excited at 350 nm and 470 nm for 810 nm and 525 nm emissions, respectively. (FIG. 4D) Normalized fluorescence intensity of FG-AuNPs and 5(6)-carboxylfluorescein at 525 nm.

FIGS. 5A-B. The chemical structure of (FIG. 5A) 5(6)-Carboxytetramethylrhodamine and (FIG. 5B) 5(6)-Carboxytetramethylrhodamine N-succinimidyl ester.

FIG. 6. Excitation spectra of TG-AuNPs collected at emission wavelengths of 630 nm and 810 nm, respectively.

FIG. 7. Fluorescence intensity ratio of TG-AuNPs at different concentrations of pH 10 to pH 7 at 590 nm and 830 nm, respectively.

FIG. 8. Fluorescence lifetime of 810 nm emission from GS-AuNPs and TG-AuNPs excited by 350 nm.

FIG. 9. Zeta potentials of TG-AuNPs as a function of pH.

FIGS. 10A-B. The chemical structure of (FIG. 10A) 5(6)-Carboxyfluorescein and (FIG. 10B) 5(6)-Carboxyfluorescein N-hydroxysuccinimide ester.

FIG. 11. Fluorescence spectra of 5(6)-carboxylfluorescein at different pHs (excitation: 470 nm).

FIG. 12. Excitation spectra of FG-AuNPs collected at emissions of 565 nm and 810 nm, respectively.

FIGS. 13A-B. Excitation and emission spectra of (FIG. 13A) 5(6)-carboxylfluorescein, GS-AuNPs and (FIG. 13B) TAMRA and GS-AuNPs, respectively, where the overlap between fluorescein emission and GS-AuNPs's excitation is 47% more than that between TAMRA's emission and GS-AuNPs' excitation.

FIG. 14. Fluorescence lifetimes of 810 nm emission from GS-AuNPs and FG-AuNPs (Excitation at 350 nm).

FIG. 15. The relationship between pH and I₅₈₇ nm/I₈₁₅ m ratios of FG-AuNPs.

FIGS. 16A-D. (FIG. 16A) High resolution transmission electron microscopy image and (FIG. 16B) size distribution of the TG-AuNPs (7 nm). (FIG. 16C) Fluorescence spectra of TG-AuNPs (7 nm) at different pH values. (FIG. 16D) Absorption spectra of TG-AuNPs (7 nm) at different pHs.

FIG. 17. Gly-Cys coated AuNPs. After conjugation, TAMRA also became pH responsive.

FIGS. 18A & 18B. (FIG. 18A) Emission spectra of TAMRA from cysteine-glycine coated gold nanoparticles (Cys-AuNPs) of 2.5 nm at different pHs. (FIG. 18B) Emission intensities of TAMRA conjugated-Cys-AuNPs over different pHs: TAMRA emission increases with the decrease of pH, which is distinct to pH responses of TG-AuNPs.

FIGS. 19A & 19B. (FIG. 19A) Emission spectra of TAMRA from cysteine-glycine coated gold nanoparticles of 2.5 nm at different pHs. (FIG. 19B) Emission intensities of TAMRA conjugated-AuNPs over different pHs: TAMRA emission increases with the decrease of pH, which is distinct to pH responses of TAMRA on glutathione coated luminescent gold nanoparticles.

FIGS. 20A & 20B. (FIG. 20A) Emission spectra of TAMRA from cysteine-glutamate coated gold nanoparticles (Cys-Glu-AuNPs) of 2.5 nm at different pHs. (FIG. 20B) Emission intensities of TAMRA conjugated- Cys-Glu-AuNPs over different pHs: TAMRA emission increases with the increase of pH.

FIG. 21. Fluorescence intensities of TAMRA on GS-AuNPs before and after being incubated with 10 mM GSH.

### DETAILED DESCRIPTION OF EXEMPLARY EMBODIMENTS

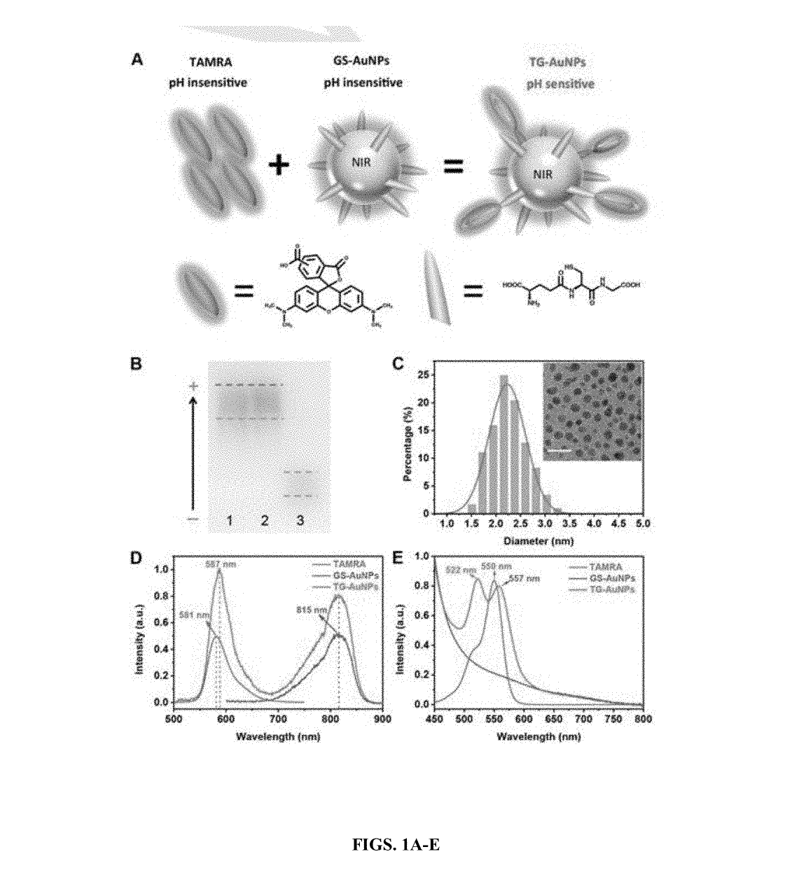

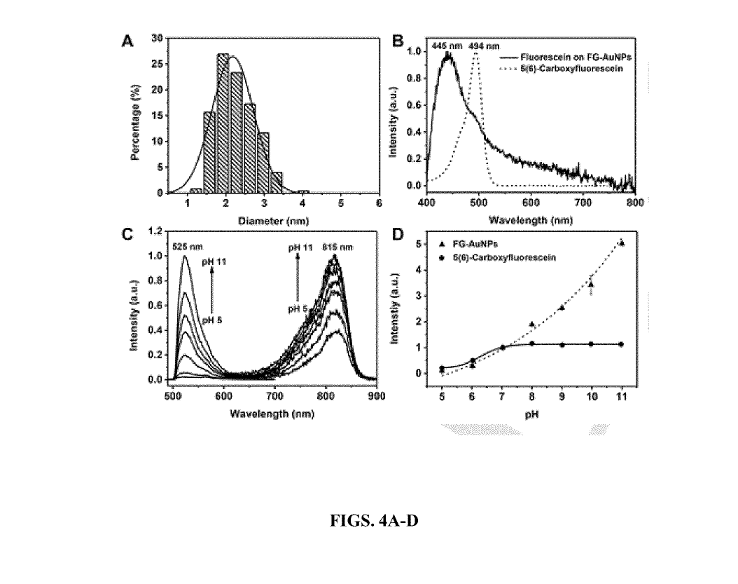

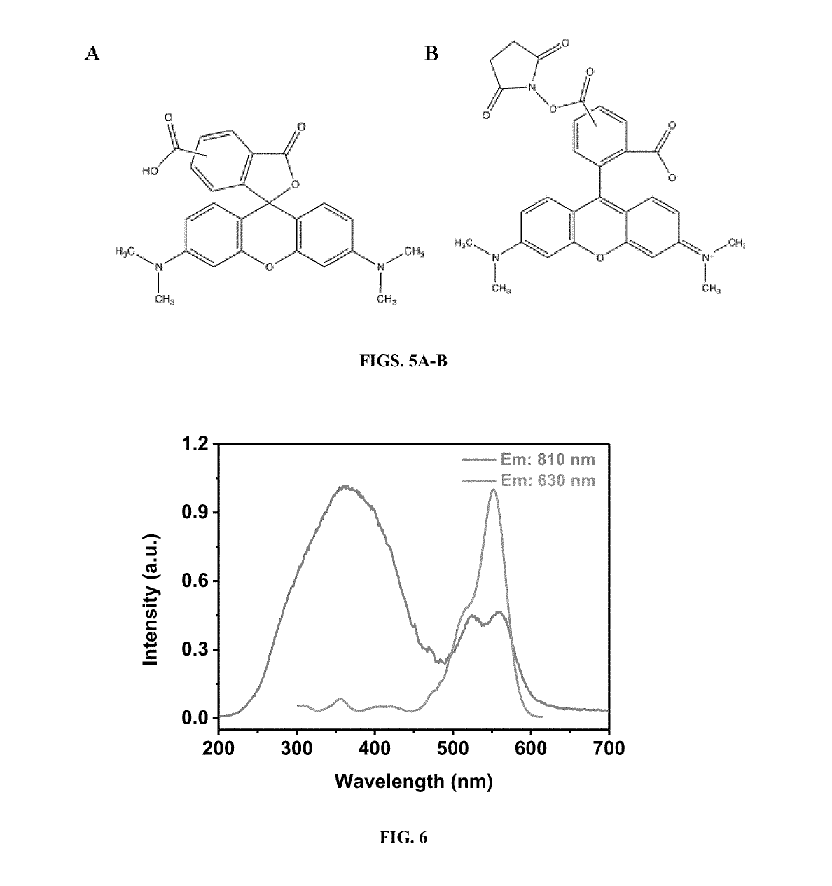

Here, the inventors report conjugating a pH insensitive dye, 581 nm emitting TAMRA (FIGS. 5A-B) onto pH insensitive near infrared emitting glutathione coated AuNPs (GS-AuNPs) (FIG. 1A), which can lead to reversible ultrasmall ratiometric pH sensors. When the pH increased from 6 to 10, the emission intensity of TAMRA increased over five-fold, while the emission intensity of GS-AuNPs only changed less than 10%. Thus, the emission ratio of TAMRA to GS-AuNP was found to linearly depend on the pH of local environment. Photophysical spectroscopic studies show that this pH dependent emission of TAMRA originates from its dimerization on the AuNPs, of which the geometry was highly sensitive to the protonation of glutathione on AuNPs. Not limited to pH insensitive dyes, a well-known pH sensitive dye, fluorescein, became even more sensitive to pH changes within a larger pH range after being conjugated to GS-AuNPs. These results clearly indicate that this unique synergy arising from conjugation of organic dyes onto luminescent AuNPs offers a new pathway to design ultrasmall ratiometric pH-responsive nanoindicators.

Additionally, these nanoparticles may include other pH sensitive surface ligands such as cysteine, cysteine-glutamate, and cysteine-glycine which may be used to synthesize the luminescent nanoparticles and the fabricated organic dye conjugated nanoparticles, which can result in distinct pH-dependent emissions.

In addition to the ability of these nanoparticles to sense pH, the nanoparticles may be used to sense glutathione, cysteine, and other thiolated compounds in the physiological environment.

These and other aspects of the disclosure are set out in detail below.

### I. Nanoparticles

The claimed disclosure provides nanoparticle compositions comprising a gold nanoparticle, methods for preparing the nanoparticle compositions and methods of using the nanoparticle compositions. As used herein, the term “nanoparticle” refers to an association of 2-1000 atoms of a metal, in this case gold. Nanoparticles may have diameters in the range of about 0.5 to about 5 nm. As used herein, diameter refers to the core size of the metal core. The metal core may be a gold core. In other preferred embodiments, the nanoparticles comprise approximately 2-1000, approximately 2-500, approximately 2-250, approximately 2-100, approximately 2-25 atoms, or approximately 2-10 atoms.

The nanoparticles may also be luminescent and emit one, two or more different colored emissions ranging from blue to IR.

The properties of the nanoparticles enable excretion through the kidneys, as well as selective uptake and retention in tumors compared with normal tissues. This, along with the lack of in vivo toxicity, has resulted in a composition that is promising for translation to the clinic. In some embodiments of the present disclosure, greater than 30%, 40%, 50%, 60%, 70%, 80% or 90% of the nanoparticles may be eliminated from the body through the urinary system.

In certain embodiments, the surface of the nanoparticle is coated with a ligand. In certain embodiments of the disclosure, the anti-fouling ligand is a zwitterionic material such as sulfobetaine methacrylate (SBMA), carboxybetaine methacrylate (CBMA), poly(carboxybetaine acrylamide) (polyCBAA) or a mixed charge material. In certain embodiments of the disclosure the ligand is glutathione, cysteine, cysteine-glycine, cysteine-glutamate, and other thiolated surface ligands which can be protonated at different pHs.

In certain embodiments, the ligand (or a further ligand) is capable of binding to at least one cellular component. The cellular component may be associated with specific cell types or having elevated levels in specific cell types, such as cancer cells or cells specific to particular tissues and organs. Accordingly, the nanoparticle can target a specific cell type, and/or provides a targeted delivery for the treatment and diagnosis of a disease. As used herein, the term “ligand” refers to a molecule or entity that can be used to identify, detect, target, monitor, or modify a physical state or condition, such as a disease state or condition. For example, a ligand may be used to detect the presence or absence of a particular receptor, expression level of a particular receptor, or metabolic levels of a particular receptor. The ligand can be, for example, a peptide, a protein, a protein fragment, a peptide hormone, a sugar (i.e., lectins), a biopolymer, a synthetic polymer, an antigen, an antibody, an antibody fragment (e.g., Fab, nanobodies), an aptamer, a virus or viral component, a receptor, a hapten, an enzyme, a hormone, a chemical compound, a pathogen, an aromatic compound, a microorganism or a component thereof, a toxin, a surface modifier, such as a surfactant to alter the surface properties or histocompatability of the nanoparticle or of an analyte when a nanoparticle associates therewith, and combinations thereof.

Other metals such as silver, copper, and platinum and even carbon, etc., may be used to generate nanoparticles for use in accordance with the present disclosure, including in ultrasmall and/or luminescent nanoparticles.

### II. Preparing Nanoparticles

The nanoparticles may be synthesized as has been reported in 8a. This approach may be used to synthesize nanoparticles such as an 815 nm GSAu-NPs, 650 nm cysteine-glycine coated gold nanoparticle (Cys-Gly-AuNPs), and 600 nm/800 nm cysteine-glutamate gold nanoparticles (Cys-Glu-AuNPs). Additionally, procedures such as thermal decomposition at room temperature may be used to prepare the nanoparticles such as 650 nm emitting cysteine-AuNPs (Cys-AuNPs).

Additionally, the near IR emitting GS-AuNPs may be prepared using the following protocol: 150 μL of 1 M HAuCl₄ solution was added to 50 mL of 2.4 mM glutathione solution in a 100 mL three-necked flask while stirring vigorously. The mixture was then heated with an oil bath at 90° C. for 35 min. The resulting solution was cooled to room temperature and centrifuged at 21,000 g for 1 min to remove large aggregates. The NPs were precipitated out of the supernatant using the following steps: adding 1 M NaOH to the supernatant to adjust the pH to approximately 3; adding ethanol solution (2:1, V_{water}/Vₑₜₕₐₙₒₗ); and centrifuging at 4,000 g for 5 min. The precipitates were suspended in 300 μL PBS buffer, and 1 M NaOH was added to adjust the pH to approximately 7. The PBS solution was centrifuged at 21,000 g for 1 min. The supernatant was the final product. For the animal studies, the GS-AuNPs were further purified using a NAP-5 column (Sephadex G-25 DNA Grade gravity columns) in phosphate buffer saline (PBS). The protocol to synthesize NIR-emitting GS-AuNPs is based upon the protocol described in Liu et al., 2013, which is incorporated herein by reference.

These noble metal nanoparticles may be prepared using noble metals or noble metal salts and then incubated with a dye in a buffer. In some embodiments, the dye has an activated group which reacts with one of the metal atoms on the surface of the nanoparticle such as a thiol group reacting with a metal ion. In some embodiments, the ratio of the noble metal nanoparticle to the dye is about 1:100 to about 1:10,000. In some embodiments, the concentration of the nanoparticle is less than 100 nM. In some embodiments, the concentration of the dye is greater than 10 nM. In some embodiments, these components are dissolved in a pH buffer. The incubation of the dye with the nanoparticle may be carried out in the dark. Without wishing to be bound by any theory, it is believed that carrying out the reaction in the dark reduces the photobleaching the dye. After incubating these components together for a period of time from about 1 hour to about 72 hours, the excess dye is removed by dialysis or chromatography. In some embodiments, the excess dye is removed via column chromatography such as with a size exclusion or Sephadex® column. The attachment of the dye may be confirmed using gel electrophoresis or other appropriate spectroscopic techniques.

### III. Dyes

The present disclosure provides for improved imaging compositions that create or increase pH sensitivity in pH insensitive and pH sensitive dyes, respectively. In some embodiments, the nanoparticles comprises two or more dyes, such as 2-100, 2-50, or 2-20 dyes per nanoparticle. A non-limiting discussion of such dyes is provided below.

A. pH Insensitive

pH insensitive dyes include tetramethyl-rhodamine (TAMRA), Alexa Fluor Dyes, boron-dipyrromethene (BODIPY), and several other dyes which show little response to the pH of the local environment.

B. pH Sensitive Dyes and Weakly pH Sensitive Dyes

pH sensitive/weakly sensitive dyes include fluorescein, 5(6)-carboxyfluorescein, 7-hydroxycourmarin-3-carboxylic acid, rhodamine B octadecyl ester perchlorate, 2′,7′-Bis(3-carboxypropyl)-5(6)-carboxyfluorescein (BCECF), 8-hydroxypyrene-1,3,6-trisulfonic acid trisodium salt (HPTS), Acid Blue 113, Brilliant Yellow, Zinc 5,10,15,20-tetra(4-pyridyl)-21H, 23H-porphine, Protoporphyrin IX di sodium salt, 5,10,15,20-tetraphenyl-21H,23H-porphine zinc low chlorin.

### IV. Methods of Use

The present disclosure further encompasses methods of using the gold nanoparticles in order to study pH of an environment or the presence of other biologically important molecules such as glutathione and cysteine. The disclosure provides for a method of detecting pH, monitoring pH change, or the presence of glutathione or cysteine in an environment over a period of time. In a particular embodiment, the environment is located in a living subject, such as a mammal, including humans. The environment may also be a site of disease, such as tumor environments, inflammation sites, and infection sites (e.g., bladder or kidney) where local pH or the concentration of glutathione or cysteine is changed dramatically.

After administration of the nanoparticle to a subject, the blood residence half-time of the nanoparticles may range from about 2 hours to about 25 hours, from about 3 hours to about 20 hours, from about 3 hours to about 15 hours, from about 4 hours to about 10 hours, or from about 5 hours to about 6 hours. Longer blood residence half-time means longer circulation, which allows more nanoparticles to accumulate at the target site in vivo. Blood residence half-time may be evaluated as follows. The nanoparticles are first administered to a subject (e.g., a mouse, a miniswine or a human). At various time points post administration, blood samples are taken to measure nanoparticle concentrations through suitable methods.

An embodiment of the claimed disclosure is directed to a gold nanoparticle that is renal clearable. In certain embodiments of the disclosure, the compositions demonstrate greater than 30% renal clearance within 48 hours of administration.

Imaging techniques employed with the gold nanoparticles described herein include optical imaging, microscopic imaging, X-ray imaging, CT imaging, SPECT imaging, PET imaging, and combinations thereof.

### V. Methods of Therapy

In an embodiment of the disclosure, a therapeutic agent may also be attached to the nanoparticle. The therapeutic agent is selected from the group consisting of antibiotics, antimicrobials, antiproliferatives, antineoplastics, antioxidants, endothelial cell growth factors, thrombin inhibitors, immunosuppressants, anti-platelet aggregation agents, collagen synthesis inhibitors, therapeutic antibodies, nitric oxide donors, antisense oligonucleotides, wound healing agents, therapeutic gene transfer constructs, extracellular matrix components, vasodialators, thrombolytics, antimetabolites, growth factor agonists, antimitotics, statin, steroids, steroidal and nonsteroidal anti-inflammatory agents, angiotensin converting enzyme (ACE) inhibitors, free radical scavengers, PPAR-γ agonists, small interfering RNA (siRNA), microRNA, and anti-cancer chemotherapeutic agents. As such, a further embodiment of the disclosure is directed to a method for targeting/treating a disease site, such as a tumor, comprising administering an effective amount of a gold nanoparticle composition as described above conjugated with at least one therapeutic agent.

### VI. Examples

The following examples are included to demonstrate preferred embodiments. It should be appreciated by those of skill in the art that the techniques disclosed in the examples that follow represent techniques discovered by the inventors to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Materials and Equipment. All chemicals were purchased from Fisher Scientific and were used as received without further purification. Transmission electron microscopy (TEM) images of the RG-AuNPs were obtained using a JEOL 2100 transmission electron microscope with a 200 kV accelerating voltage. Hydrodynamic diameters (HDs) of the samples in the aqueous solution were analyzed using a Brookhaven 90Plus Dynamic Light Scattering (DLS) Particle Size Analyzer. Zeta potentials of the samples were analyzed by a Brookhaven ZetaPALS zeta potential analyzer. Fluorescence lifetimes were measured with a PTI time resolved fluorescence lifetime spectrometer. pH was measured by a Accumet AB15 pH meter and a Accuphast microprobe electrode. Absorption spectra were taken using a Varian 50 Bio UV-Vis spectrophotometer.

Synthesis method and characterization—Synthesis of TG-AuNPs. To conjugate organic dyes including TAMRA and Fluorescein to GS-AuNPs, Cys-AuNPs, Cysgly-AuNPs, NHS- TAMRA solution was added to GS-AuNPs in phosphate buffered saline (PBS) solution in a vial. The mixture was shaken at room temperature in the dark for 24 h. The NHS ester group of TAMRA reacted with the amines on glutathione to form a stable amide bond. After the reaction was completed, the mixture was further purified to remove the unreacted TAMRA molecules.

Quantification of the number of TAMRA per nanoparticle. To quantify the number of dye molecules per individual AuNP, a standard calibration curve of TAMRA absorbance as a function of TAMRA concentration was made in the range of Beer's law. The TG-AuNPs solution was treated with 10 mM glutathione solution for 24 h. Then, the amount of 5(6)-TAMRA in the solution was determined using UV-Vis and the concentration of gold in the solution was quantified via ICP-Mass Spectroscopy. With these studies, the number of dye molecules per GS-AuNP were in the range from 2-20.

### Example 2

### Results

Synthesis of TAMRA conjugated GS-AuNPs (TG-AuNPs) was very straightforward. The inventors synthesized 815 nm emitting GS-AuNPs with a reported method before (Liu et al., 2013) and then incubated NHS-TAMRA with GS-AuNPs in phosphate buffered saline (PBS) solution in the dark at room temperature. The conjugation of TAMRA onto GS-AuNPs was first confirmed by agarose gel electrophoresis. As shown in FIG. 1B, while yellowish GS-AuNPs (1) and free pinkish TAMRA (3) have distinct motilities, reddish-colored TG-AuNPs (2) have a mobility similar to that of GS-AuNPs. High Resolution Transmission Electron Microscopy (HR-TEM) showed that the average diameter of the TG-AuNPs is 2.2±0.4 nm (FIG. 1C), consistent with the core size (2.1±0.5 nm) of GS-AuNPs (Liu et al., 2013). The average number of TAMRA on one GS-AuNP was found to be about 4 (see the methods described above).

While AuNPs are known to partially quench fluorescence of organic dyes on their surface^{[9]}, fluorescence spectra of TG-AuNPs showed that dual-colored emissions were still observed and centered at 587 nm and 815 nm under 350 nm excitation, corresponding to the emissions of TAMRA dyes and GS-AuNPs, respectively (FIG. 1D and FIG. 6 (the detailed excitation spectra)). Compared to the emission maximum (581 nm) of free monomeric TAMRA in aqueous solution, a 6 nm red shift in the emission of TAMRA after being conjugated onto GS-AuNPs implies dipole-dipole couplings among TAMRA molecules on AuNPs (Del Monte et al., 2000), which was further confirmed by their UV-Vis absorption. As shown in FIG. 1E, free TAMRA exhibit a strong absorption peak at 550 nm. An additional small shoulder peak at 522 nm was due to association between monomeric TAMRA molecules in aqueous solution (Ogawa et al., 2010). After the conjugation to GS-AuNPs, the strong absorption at 550 nm was split into two peaks: one peak was located at 522 nm and the other was red shifted to 557 nm, consistent with previous reports (Valdesaguilera and Neckers, 1989; Ogawa et al., 2009; Kobayashi et al., 2010) on dimerization of TAMRA molecules, further confirming dipole-dipole couplings of TAMRA dimers on GS-AuNPs. Since both blue and red shifts in absorption of TAMRA were observed, according to well-known molecular excitation coupling theory (Valdesaguilera and Neckers, 1989), the dimers of TAMRA are not parallel to each other but have intermediate geometries (Adachi et al., 2014).

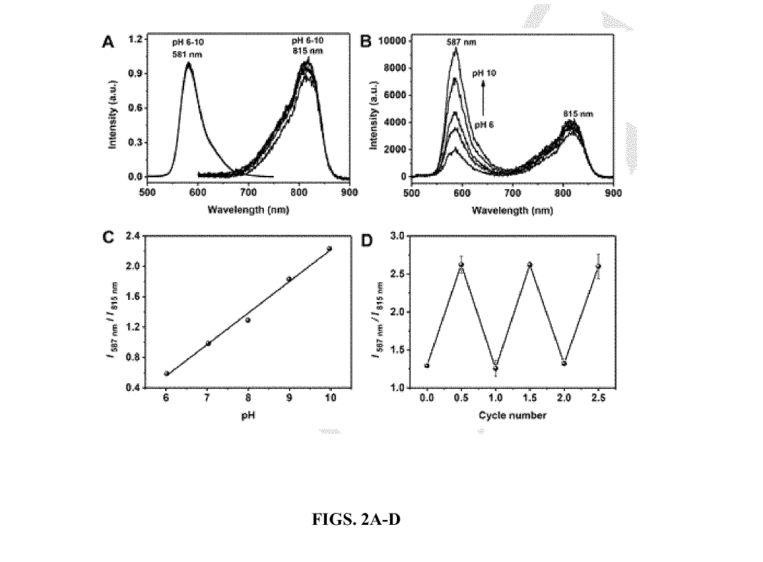

While both monomeric TAMRA and GS-AuNPs are insensitive to pH changes from 6 to 10 (FIG. 2A), TAMRA exhibited strong pH-dependent emission after being conjugated onto GS-AuNPs. As shown in FIG. 2B, with the increase of the pH value from 6 to 10, the fluorescence intensity at 587 nm gradually increased for more than 5 times while the emission intensity from the GS-AuNPs changed less than 10% in this range. Thus, the intensity ratio of the two emissions became linearly dependent of pH (FIG. 2C). Such dependence is highly reversible. As shown in FIG. 2D, by repeating switching pH from 6 to 10, very little variation in the ratio, which is different from those irreversible pH sensors derived from micelle or avidin loaded with TARMA (Ogawa et al., 2010; Zhou et al., 2011) and other known ratiometric pH indicators (Hanson et al., 2004; Kurishita et al., 2010; Dennis et al., 2012). The pH response of TG-AuNPs was still independent of the concentration (FIG. 7), indicating the changes in the fluorescence intensity at different values were not induced by the aggregation of AuNPs.

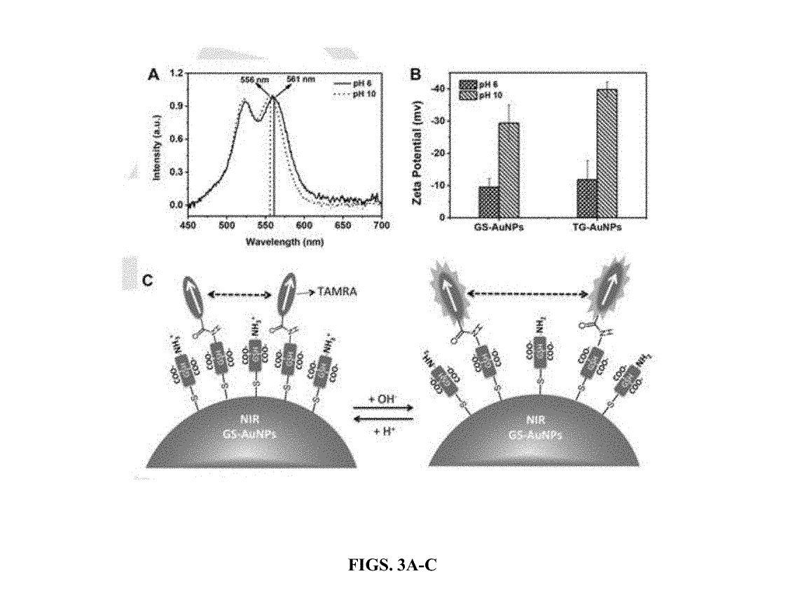

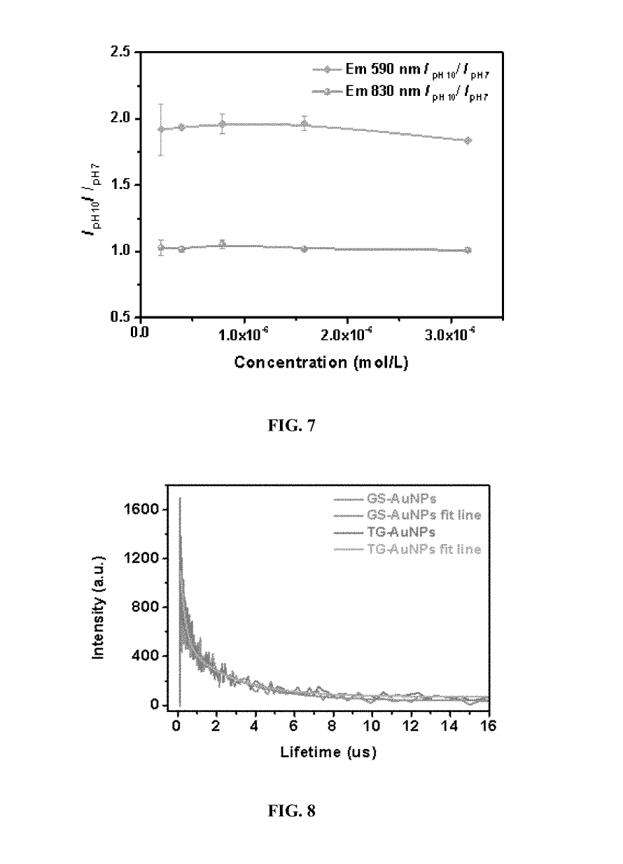

To fundamentally understand the pH response mechanism of TAMRA on GS-AuNPs, the inventors further investigated UV-Vis absorption of TG-AuNPs at pH 6 and 10, respectively. As shown in FIG. 3A, with the decrease of pH from 10 to 6, the absorption peak at 522 nm only showed very little red shift (˜0.6 nm) while the absorption peak at 556 nm was red shifted to 561 nm. This suggested that the pH did not significantly affect head-to-head coupling between transition dipoles of dimers; however, it did significantly strengthen the head-to-tail coupling between transition dipoles. These changes also implied that the angle between two transition dipoles became larger and the couplings were weaken with pH increase (Adachi et al., 2014). Therefore, energy transfer between TAMRA dimers became less efficient and emission of TARMA dramatically increased (Valdesaguilera and Neckers, 1989; Ogawa et al., 2009; Adachi et al., 2014). On the other hand, 810 nm emission of GS-AuNPs shows very little pH response (<10%), similar to pure GS-AuNPs, implying little fluorescence resonance energy transfer between TAMRA and GS-AuNPs. This result is consistent with our fluorescence lifetime measurements. The average lifetime of 810 nm emission from pure GS-AuNPs was measured to be 1.39±0.08 μs, nearly identical to that (1.19±0.05 μs) from TG-AuNPs, indicating no fluorescence energy transfer between TAMRA and GS-AuNPs (FIG. 8).

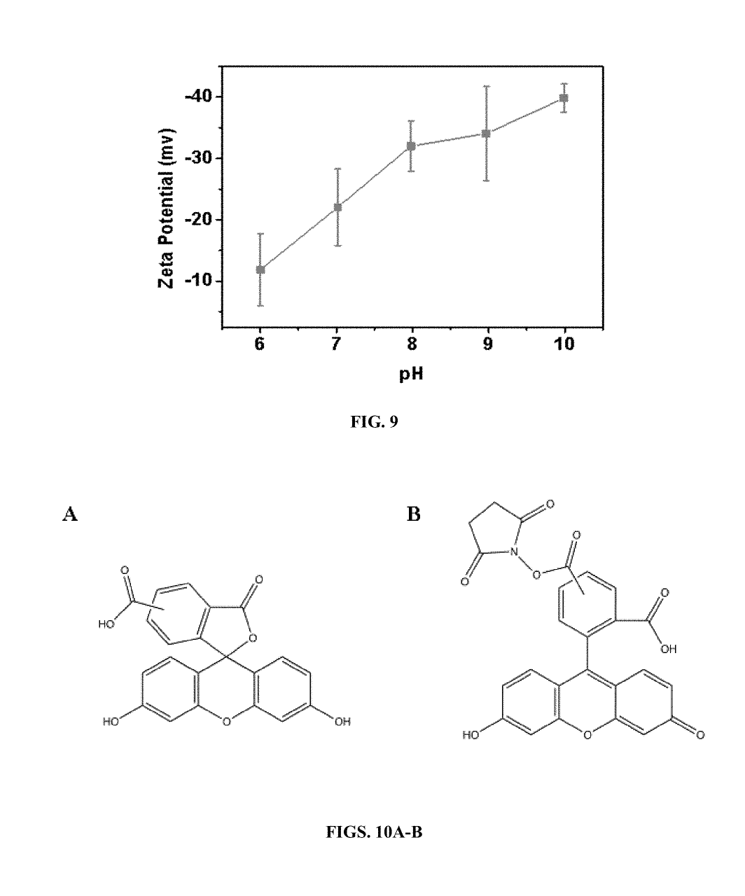

To further understand the origin of the pH effect on the dimerization of TAMRA on GS-AuNPs, the inventors measured the zeta potentials of TG-AuNPs at pH 10 and 6. As shown in FIG. 3B, with the decrease of pH from 10 to 6, the overall zeta potential of TG-AuNPs was reduced by about −20 mV (from −39.8 mV to −11.8 mV), almost identical to the zeta potential change (from −29.4 my to −9.5 mV) of pure GS-AuNPs (Yu, et al., 2011). The reduction in zeta potential is attributed to the protonation of amine group of GS-AuNPs rather than TAMRA because TG-AuNPs and pure GS-AuNPs showed an almost identical trend. More detailed studies on zeta potential of TG-AuNPs at different pH conditions showed that the decrease of pH resulted in gradual reduction on the overall surface charge of TG-AuNPs due to protonation of amine group of glutathione (FIG. 9). The observed gradual decrease in the surface charges due to protonation of amine group of glutathione immobilized on the AuNPs is consistent with recent reports on pKa changes of these amine or carboxylate groups after being immobilized on NP surface (Wang et al., 2011). Combining the UV-Vis absorption, fluorescence and zeta potentials of TG-AuNPs at different pH environments, a hypothesized mechanism was proposed in FIG. 3C: with the increase of pH from 6 to 10, the angle between transition dipoles of TAMRA dimers on GS-AuNPs was slightly increased and the couplings of TAMRA dimers was reduced due to the increased repulsion among negatively charged GSH ligands on the AuNPs. Thus, the fluorescence intensity of TAMRA would dramatically increase with pH increase.

To investigate whether the observed reversible pH-dependent dimerization can be applied to pH sensitive dyes, the inventors conjugated 5(6)-carboxyfluorescein (FIG. 10A), a well-known pH sensitive dye (Han and Burgess, 2009), onto GS-AuNPs using the same method (see SI for the details). Average size of fluorescein-conjugated GS-AuNPs (FG-AuNPs) is 2.2±0.5 nm (FIG. 4A), similar to TG-AuNPs. After subtracting the absorption of GS-AuNPs, a new peak at 445 nm was observed from FG-AuNPs, 47 nm blue shifted compared to the absorption maximum of free fluorescein (FIG. 4B) and consistent with the previous report (˜40 nm) on H-dimerization of fluorescein^{[19]}.

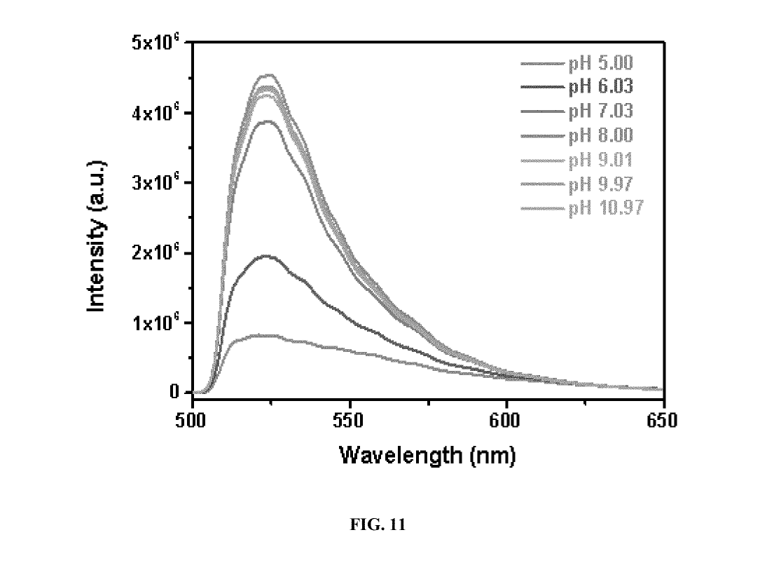

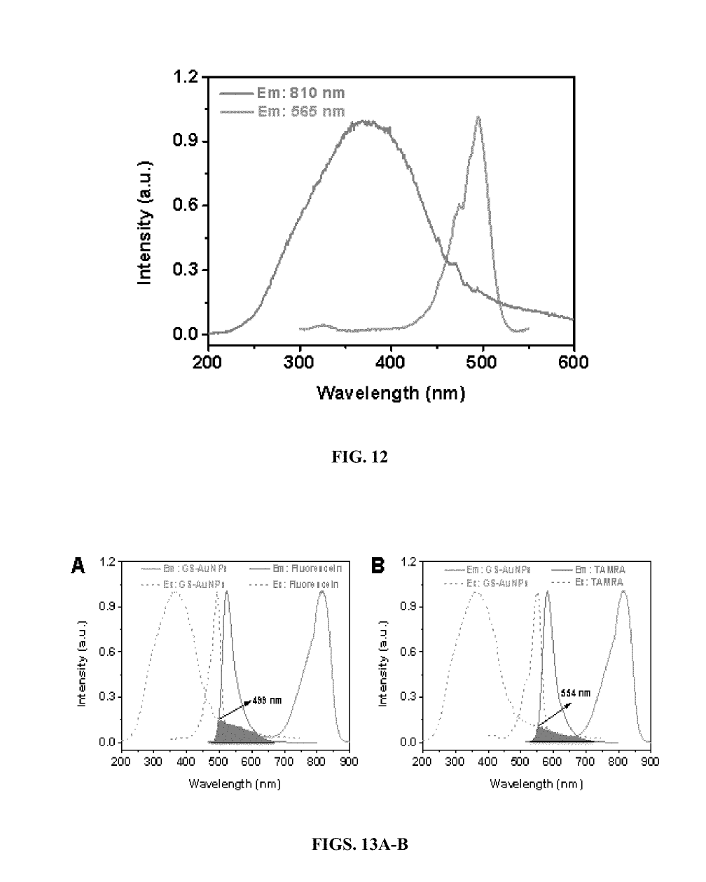

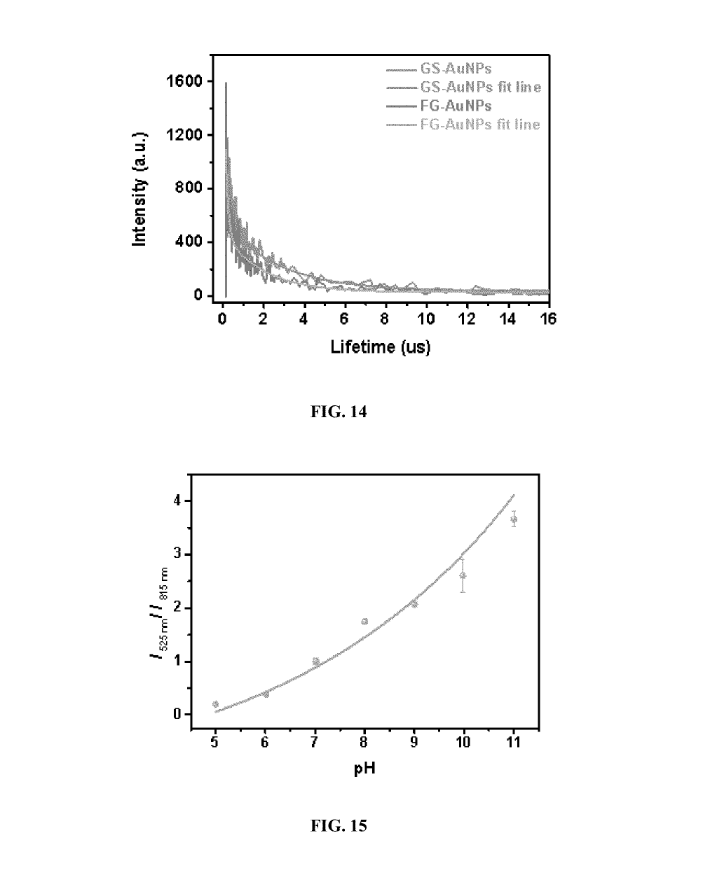

A much more sensitive pH-dependent emission was observed in FG-AuNPs. When pH is increased from 5 to 11, the 525 nm emission intensity of fluorescein on AuNPs under 470 nm excitation had increased about 19 times (FIG. 4C,); it was nearly ˜4 times more sensitive to pH than that of free fluorescein in the same pH window (FIG. 4D). In addition, unlike free fluorescein that only responded to pH change in the range of 5 to 7 (FIG. 11), the emission of fluorescein on AuNPs remained pH responsive until the pH reached 11. More interestingly, 815 nm emission of AuNPs after fluorescein conjugation (under 350 nm excitation) (FIG. 12) became sensitive to pH changes: as the pH increases from 5 to 11, the 815 nm emission increased about 3.5 times. This difference between TG-AuNPs and FG-AuNPs is likely due to the more efficient energy transfer between fluorescein emission and GS-AuNPs excitation, resulted from a larger spectral overlap (47% more) (FIGS. 13A-B). To further confirm energy transfer between fluorescein and GS-AuNPs, the inventors also measured the lifetimes of 810 nm emission from GS-AuNPs before and after conjugated with fluorescein and found that the lifetime of 810 nm decreased from 1.39±0.08 μs to 0.81±0.05 μs (FIG. 14), different from the observation of TG-AuNPs (FIG. 15), further confirming energy transfer between fluorescein and GS-AuNPs. Moreover, the FG-AuNPs can also report pH in a ratiometric way (FIG. 15).

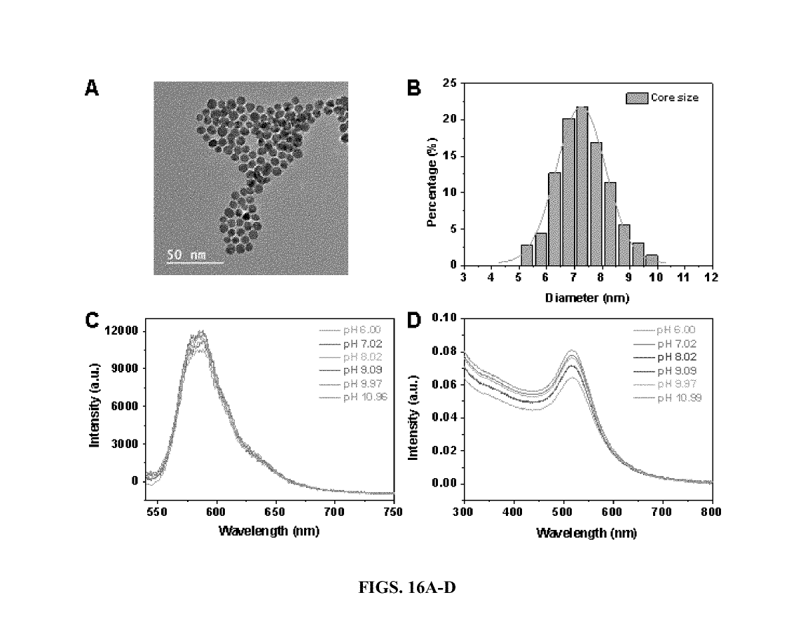

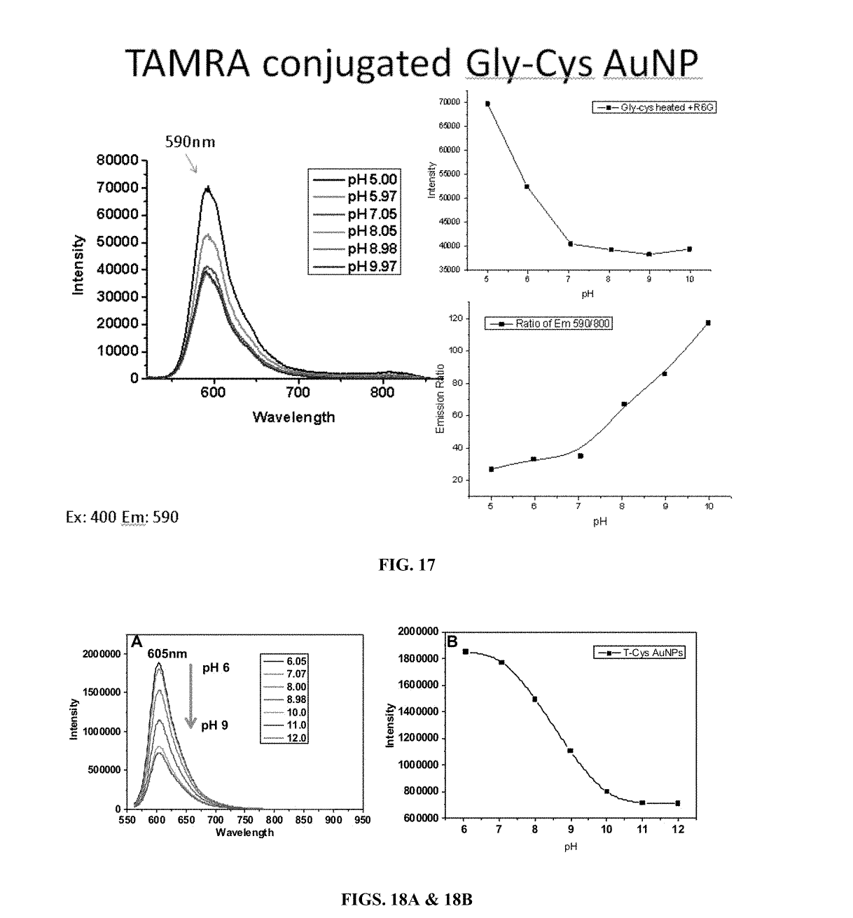

It should be noted that such pH-dependent emission is limited to ultrasmall AuNPs because the pH-dependent emission was no longer observed from TAMRA on the 7 nm TG-AuNPs surface even though TAMRA emission was still observed (FIGS. 16A-D). Nevertheless, this synergistic effect resulted from simple conjugation of organic dyes to ultrasmall luminescent AuNPs opens up a new pathway to design fluorescent ratiometric nanoindicators with tunable emission wavelengths and a broad pH responsive range.

Not limited to glutathione coated luminescent gold nanoparticles, the similar strategy can also be used to conjugate organic dyes to luminescent gold nanoparticles coated by cysteine. For example, TAMRA conjugated cysteine coated luminescent AuNPs (Cys-AuNPs) also exhibit pH responsive emissions. Shown in FIG. 18A are emission spectra of TAMRA conjugated on AuNPs. With the decrease of pH from 9 to 6, the emission intensity was increased, which is distinct to pH responses from TAMRA conjugated on GS-AuNPs. As shown in FIG. 2B, with the increase of the pH value from 6 to 10, the fluorescence intensity of TAMRA gradually increased rather than the decrease. These results clearly indicated that the same organic dye on luminescent gold nanoparticles can exhibit reverse pH responses upon the surface ligands.

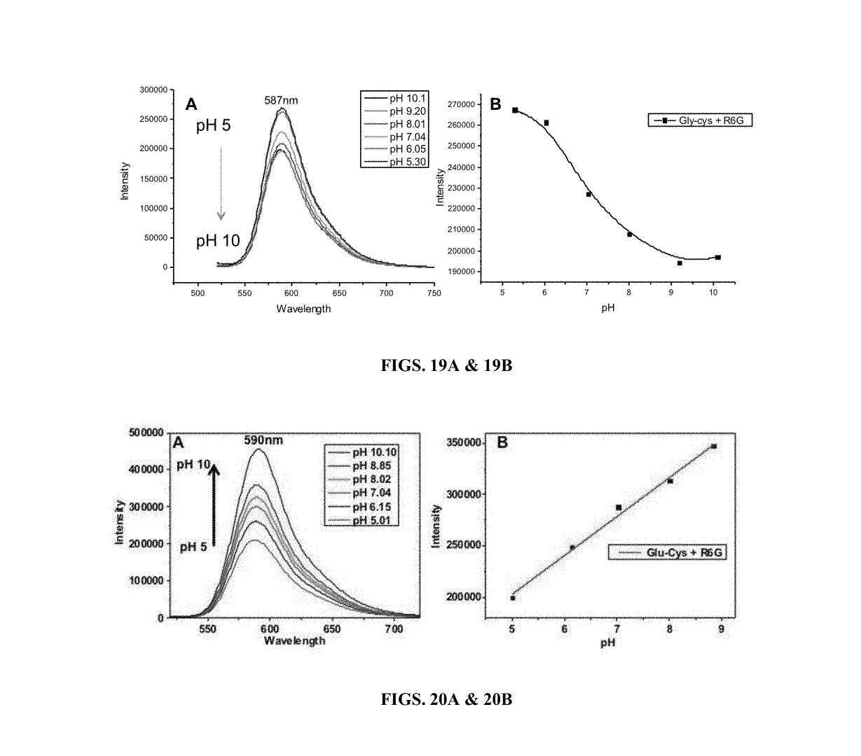

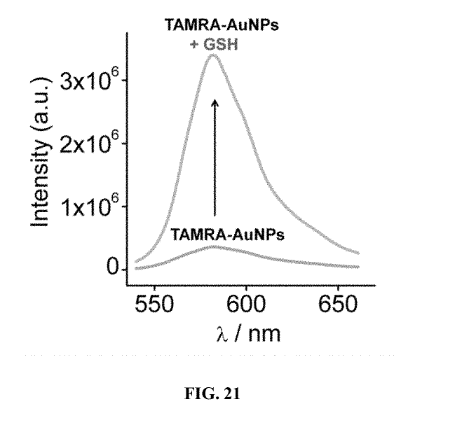

To further demonstrate that pH responses from organic dyes can be tuned by tuning other surface ligands, cysteine-glycine coated luminescent AuNPs (Cys-gly-AuNPs) and cysteine-glutamate coated luminescent AuNPs (Cys-glu-AuNPs) were also synthesized using the procedure reported before, followed by conjugating TAMRA onto these two AuNPs. Shown in FIGS. 19A & 19B are emission spectra of TAMRA on Cys-gly-AuNPs at different pHs and emission intensities were also linearly increased with the decrease of pHs from 9 to 5. Slightly changing the surface ligand from cysteine-glycine to cysteine-glutamate, the pH responses of TAMRA on Cys-glu-AuNPs were completely reversed: with the decrease of pH from 10 to 5, the emission was linearly decreased (FIG. 20).

Combination of all the results presented above, we found that conjugation of organic dyes to ultrasmall luminescent AuNPs leads to a unique synergy that enables pH insensitive dyes to exhibit pH-dependent emission and pH sensitive dyes to become more sensitive to pH changes in a larger pH range. Therefore, the emission ratio of organic dyes and AuNPs can be used to quantitatively report the local pH changes. Such intriguing pH responses originate from the dimerization of organic dyes on the ultrasmall AuNP surface, of which geometries were found to be very sensitive to surface charges and can be modulated through the protonation of surrounding AuNP surface ligands. It should be noted that such pH-dependent emission is limited to ultrasmall AuNPs because the inventors no longer observed similar pH-dependent emission from TAMRA on the 7 nm TG-AuNPs surface even though TAMRA emission was still observed (FIGS. 16A-D). Nevertheless, this synergistic effect resulted from simple conjugation of organic dyes to ultrasmall luminescent AuNPs opens up a new pathway to design fluorescent ratiometric nanoindicators with tunable emission wavelengths and a broad pH responsive range.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. References

The following references, to the extent that they provide exemplary procedural or other details supplementary to those set forth herein, are specifically incorporated herein by reference.

Adachi et al., Ind. Eng. Chem. Res., 53, 13046-13057; 2014.

Chen et al., J. Am. Chem. Soc., 135, 11595-11602, 2013.

Chou and Chan, Adv. Healthcare. Mater., 1, 714-721, 2012.

Del Monte et al., Langmuir, 16, 7377-7382; 2000.

Dennis et al., ACS Nano, 6, 2917-2924; 2012.

Han and Burgess, Chem. Rev., 110, 2709-2728; 2009.

Hanson et al., Biol. Chem., 279, 13044-13053; 2004.

Hu and Gao, ACS Nano, 4, 6080-6086, 2010.

Kobayashi et al., Chem. Rev.,110, 2620-2640; 2010.

Kurishita et al., J. Am. Chem. Soc., 132, 13290-13299; 2010.

Liu et al., Angew. Chem.; 52, 12572-12576, 2013.

Ogawa et al., Acs Chemical Biology, 4, 535-546; 2009.

Ogawa et al., Mol. Biosyst., 6, 888-893; 2010.

Palner et al., Angew. Chem., 54, 11477-11480; 2015.

Valdesaguilera and Neckers, Accounts Chem. Res., 22, 171-177; 1989.

Wang et al., J. Am. Chem. Soc., 133, 2192-2197; 2011.

Wei et al., Analyst., 135, 1406-1410; 2010.

Xie et al., Biomaterials, 31, 3016-3022; 2010.

Yu and Zheng, ACS Nano, 9, 6655-6674; 2015.

Yu et al., Angew. Chem., 54, 15434-15438; 2015.

Yu et al., J. Am. Chem. Soc., 133, 11014-11017, 2011.

Zheng et al., J. Am. Chem. Soc., 125, 7780-7781; 2003.

Zheng et al., Annu. Rev. Phys. Chem., 58, 409-431, 2007.

Zheng et al., Nanoscale, 4, 4073, 2012.

Zheng et al., Phys. Rev. Lett., 93, 077402; 2004.

Zhou et al., Angew. Chem., 50, 3168-3172; 2011.

Zhou et al., Angew. Chem., 50, 6109-6114; 2011.

Zhou et al., Angew. Chem., 51, 10118-10122; 2012.

1. A composition comprising a noble metal nanoparticle, wherein the surface of the nanoparticle is conjugated with (a) a charged ligand susceptible to protonation or deprotonation by pH change and (b) a dye that is pH-insensitive when not conjugated with the noble metal nanoparticle.

2. The composition of claim 1, wherein the noble metal is gold.

3. The composition of claim 1, wherein the nanoparticle is about 0.5 nm to 10 nm in diameter, or 1 nm to 5 nm in diameter.

4. The composition of claim 1, wherein the nanoparticle is luminescent.

5.-6. (canceled)

7. The composition of claim 1, wherein the charged ligand is glutathione, cysteine, cysteine-glycine, cysteine-glutamate, or any other ligand that is differentially protonated across various pHs.

8.-13. (canceled)

14. The composition of claim 1, wherein the composition comprises two or more pH insensitive dye molecules.

15.-16. (canceled)

17. The composition of claim 1, wherein the nanoparticle is sensitive to a pH from about 5 to about 11, from about 6 to about 10, from about 7 to about 9, or at about pH 5, 6, 7, 8, 9, 10 or 11.

18. The composition of claim 1, noble metal may consist of, comprise, or consist essentially of silver, copper, platinum, or carbon, and optionally is luminescent.

19. The composition of claim 18, wherein the size of the nanoparticle is less than 3 nm.

20. A method for detecting pH of an environment comprising the steps of:
(a) contacting the environment with a composition comprising a nanoparticle of claim 1; and
(b) monitoring the emission from the nanoparticle by optical imaging, microscopic imaging or combinations thereof.

21.-24. (canceled)

25. A composition comprising a noble metal nanoparticle, wherein the surface of the nanoparticle is conjugated with (a) a charged ligand susceptible to protonation or deprotonation by pH change and (b) a pH-sensitive dye, wherein said pH-sensitive dye is more pH sensitive when conjugated to said nanoparticle than when not conjugated with the nanoparticle.

26. The composition of claim 25, wherein the nanoparticle is about 0.5 nm to 10 nm, or about 1 nm to 5 nm in diameter.

27. The composition of claim 25, wherein noble metal may consist of, comprise, or consist essentially of gold, silver, copper, platinum, or carbon, and optionally is luminescent.

28.-30. (canceled)

31. The composition of claim 25, wherein the charged ligand is glutathione, cysteine, cysteine-glycine, cysteine-glutamate or any other ligand that is differentially protonated across various pHs.

32.-35. (canceled)

36. The composition of claim 25, wherein the pH sensitive dye is 2-fold, 3-fold, 4-fold, 5-fold or 10-fold more sensitive when conjugated to nanoparticle as compared to its unconjugated state.

37. (canceled)

38. The composition of claim 25, wherein the nanoparticle is sensitive to a pH from about 5 to about 11, from about 6 to about 10, from about 7 to about 9, or at about pH 5, 6, 7, 8, 9, 10 or 11.

39. The composition of claim 25, wherein the nanoparticle is 0.5-5 nm in diameter, and contains at least 2 dye particles.

40. A method for detecting pH of an environment comprising the steps of:
(a) contacting the environment with a composition comprising a nanoparticle according to claim 25; and
(b) monitoring the emission from the nanoparticle by optical imaging, microscopic imaging or combinations thereof.

41.-45. (canceled)

46. A method for determining the presence of a thiolated compound in an environment comprising the steps of:
(a) contacting the environment with a composition comprising a nanoparticle according to claim 1; and
(b) monitoring the emission from the nanoparticle by optical imaging, microscopic imaging, or combinations thereof, wherein a change in the emission is associated with the presence of the thiolated compound.

47.-49. (canceled)

50. The method of claim 46, wherein monitoring occurs over time and detects a change in the concentration of the thiolated compound in the environment.

51. (canceled)
